(19) United States
(12) Patent Application Publication
(10) Pub. No.: US 2024/0009648 A1
(43) Pub. Date: Jan. 11, 2024

(54) SYSTEMS AND METHODS FOR PROCESSING SHALE GAS

(71) Applicant: University of Notre Dame du Lac, South Bend, IN (US)

(72) Inventors: Jason Christopher Hicks, South Bend, IN (US); Deanna Poirier, South Bend, IN (US); Gerardo Rivera-Castro, South Bend, IN (US)

(21) Appl. No.: 18/345,866

(22) Filed: Jun. 30, 2023

Related U.S. Application Data
(60) Provisional application No. 63/367,646, filed on Jul. 5, 2022.

(51) Int. Cl. B01J 19/08; C01B 21/06; C07C 253/24; C07C 209/02; C07C 2/76; C01C 1/04

(52) U.S. Cl. CPC B01J 19/088; C01B 21/0605; C07C 253/24; C07C 209/02; C07C 2/76; C01C 1/04

(57) ABSTRACT

Shale processing systems may include a reactor comprising. a first inlet in fluid communication with a shale gas source. a plasma zone in fluid communication with the first inlet, the plasma zone comprising, an outlet in fluid communication with the plasma zone, a collection vessel configured to receive fluid from the reactor outlet, and a voltage supply and monitor system in electrical communication with the inner electrode and with the outer electrode. The shale gas processing systems may be configured to generate various fluid products, including nitrogen (N)-containing compounds.

### CROSS-REFERENCE TO RELATED APPLICATION(S)

This application claims priority to U.S. Provisional Patent Application No. 63/367,646 filed on Jul. 5, 2022, the entire contents of which are incorporated herein by reference.

### STATEMENT OF GOVERNMENT INTEREST

This invention was made with government support under DE-FE0031862 awarded by the U.S Department of Energy (DOE). The government has certain rights in the invention.

### TECHNICAL FIELD

The present disclosure relates to materials, methods, and techniques for processing shale gas. Exemplary shale gas processing systems and methods may generate various nitrogen (N)-containing compounds.

### INTRODUCTION

Chemicals containing carbon-nitrogen (C—N) bonds are highly important. However, the formation of these bonds can be difficult and often requires expensive catalyst and precursor materials, high temperatures and pressures, and hazardous chemicals, such as ammonia (NH₃). The production of hydrogen cyanide (HCN) demonstrates the complexity of C—N bond formation; for HCN synthesis, NH₃ is reacted with CH₄ over a platinum catalyst at high temperatures (1500K). The instant disclosure is directed to methods for forming nitrogen (N)-containing compounds.

### SUMMARY

The present disclosure relates to systems and methods for processing shale gas. In one aspect, a system for processing shale gas is disclosed. The shale gas processing system may comprise a reactor comprising a first inlet in fluid communication with a shale gas source, a plasma zone in fluid communication with the first inlet, an outlet in fluid communication with the plasma zone, a collection vessel configured to receive fluid from the reactor outlet, and a voltage supply and monitor system in electrical communication with the inner electrode and with the outer electrode. The plasma zone may comprise an inner electrode, an outer electrode, an inner volume defined between the inner electrode and the outer electrode. The reactor may further comprise a second inlet in fluid communication with a nitrogen (N₂) gas source. The first inlet and the second inlet may be positioned at an upper portion of the reactor; and the outlet may be positioned at a lower portion of the reactor. The system may further comprise a depressurization unit arranged to provide shale gas to the first inlet. The plasma zone may have a discharge gap of 0.1 mm to 150 mm. The plasma zone may be cylindrical, and the outer electrode may annularly define an exterior of the plasma zone. The inner electrode may comprise tungsten and the outer electrode may comprise stainless steel. The reactor may further comprise a reactor temperature regulation unit arranged to maintain a predetermined temperature within the reactor. The reactor may not include catalyst material. The system may further comprise a gas chromatograph (GC) in fluid communication with the reactor outlet. The gas chromatograph (GC) may comprise a thermal conductivity detector (TCD), a flame ionization detector (FID), and a photoionization detector (PID). The voltage supply and monitor system may comprise an alternating current (AC) power source in electrical communication with the inner electrode, an oscilloscope, a voltage attenuator in electrical communication with the AC power source and the oscilloscope, and a monitor capacitor in electrical communication with the outer electrode and the oscilloscope.

In another aspect, a method for processing shale gas is disclosed. The method for processing shale gas may comprise providing shale gas at a first flowrate to a first inlet of a reactor, providing a voltage to an inner electrode disposed within a plasma zone of the reactor, thereby generating a plasma in the plasma zone across a discharge gap, and collecting products from an outlet of the reactor. The method may further comprise providing nitrogen (N₂) gas at a second flowrate to a second inlet of the reactor. The first flowrate may be 5 standard cubic centimeters per minute (cm³/min) to 11,304 cm³/min. The second flowrate may be 1 cm min to 10,174 cm³/min. Before providing the shale gas to the first inlet of the reactor, the shale gas may be decompressed to a pressure no greater than 0.3 Megapascal (MPa). The plasma zone may be maintained at a temperature of 25° C. to 250° C. The discharge gap may be 0.1 mm to 150 mm. The voltage provided may be no less than 6 kV and no greater than 9 kV. The voltage may be provided at a frequency of 2 kHz to 700 kHz. The voltage may be provided from an AC power source. Before providing the voltage, an output signal from the AC power source may be attenuated. While providing the voltage, the voltage across a capacitor may be monitored. After collecting the products, unreacted gases may be recycled back to the first inlet of the reactor.

In another aspect, a method of preparing nitrogen (N)-containing compounds is disclosed. The method may comprise providing a gas composition to a reactor, providing a voltage to an inner electrode disposed within a plasma zone of the reactor, thereby generating a plasma in the plasma zone across a discharge gap, and collecting products from an outlet of the reactor, the products comprising nitrogen (N)-containing compounds. The gas composition may comprise, by mol %: 7% to 94% methane (CH₄), 2% to 20% ethane (C₂H₆), 1% to 11% propane (C₃H₈), and 2% to 90% N₂. The nitrogen (N)-containing compounds may comprise a combination of nitrogen (N), carbon (C), and hydrogen (H) atoms, or a combination of nitrogen (N) and hydrogen (H) atoms.

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

### BRIEF DESCRIPTION OF THE DRAWINGS

FIG. 1 is a schematic depiction of an exemplary shale processing system.

FIG. 2 is a schematic depiction of an exemplary voltage supply and monitor system for exemplary shale processing systems.

FIG. 3 shows a flowchart of an exemplary method for processing shale gas.

FIG. 4 depicts an exemplary dielectric barrier discharge reactor with liquid collection vessel.

FIG. 5 is an attenuated total reflectance Fourier transform infrared (ATR-FTIR) spectrum of the liquid product from reactions with various amounts of nitrogen in the feed gas composition.

FIG. 6 is an attenuated total reflectance Fourier transform infrared (ATR-FTIR) spectrum of the liquid product from reactions at various powers.

FIG. 7 is a matrix-assisted laser desorption ionization-time-of-flight mass spectrum (MALDI-TOF MS) of the liquid product.

FIGS. 8A-8C tabularly show 250 different exemplary products from an exemplary reaction with a feed gas composition of35 mol % CH₄, 10 mol % C₂H₆, 5 mol % C₃H₈, and 50 mol % N₂, and the products' corresponding mass divided by charge numbers (m/z values), atomic compositions (“Comp.”), and peak intensities (“intensity”), as observed by High resolution electrospray ionization mass spectrometry (ESI-MS).

FIGS. 9A-9C tabularly show 250 different exemplary products from an exemplary reaction with a feed gas composition of 7 mol % CH₄, 2 mol % C₂H₆, 1 mol % C₃H₈, and 90 mol % N₂, and the products' corresponding mass divided by charge numbers (m/z values), atomic compositions (“Comp.”), and peak intensities (“intensity”), as observed by High resolution electrospray ionization mass spectrometry (ESI-MS).

### DETAILED DESCRIPTION

Exemplary materials, methods and techniques disclosed and contemplated herein generally relate to shale gas processing systems and methods. Exemplary shale gas processing systems may be configured to process shale gas and generate fluid products. Exemplary shale gas processing methods described herein may generate various nitrogen (N)-containing compounds.

### I. DEFINITIONS

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not

As used herein, the term “about” is used to indicate that exact values are not necessarily attainable. Therefore, the term “about” is used to indicate this uncertainty limit. The term “about” may refer to plus or minus 10% of the indicated number. For example, “about 10%” may indicate a range of 9% to 11%, and “about 1” may mean from 0.9-1.1. Other meanings of “ab out” may be apparent from the context, such as rounding off, so, for example “about 1” may also mean from 0.5-1.4. The modifier “about” should also be considered as disclosing the range defined by the absolute values of the two endpoints. For example, the expression “from about 2 to about 4” also discloses the range “from 2 to 4.”

For the recitation of numeric ranges herein, each intervening number there between with the same degree of precision is contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are contemplated. For another example, when a pressure range is described as being between ambient pressure and another pressure, a pressure that is ambient pressure is expressly contemplated.

The term “alkene” as used herein, means a straight or branched, hydrocarbon containing at least one carbon-carbon double bond. The term “C₂₋₄ alkene” means a straight or branched chain hydrocarbon containing from 2 to 4 carbon atoms and at least one carbon-carbon double bond.

The term “alkyne” as used herein, means a straight or branched, hydrocarbon containing at least one carbon-carbon triple bond.

The term “non-thermal plasma” as used herein, means a plasma which is not in thermodynamic equilibrium with the immediate environment. “Non-thermal plasma” is alternatively referred to in literature as “cold plasma” or “non-equilibrium plasma.”

### II. EXEMPLARY MATERIALS

Exemplary methods and techniques use and process various materials. Exemplary shale gas processing materials include gas compositions and products. Various aspects of exemplary gas compositions and products are discussed below.

### A. Exemplary Gas Compositions

Exemplary gas compositions include shale gases. Exemplary shale gases may comprise methane (CH₄). In various instances, exemplary shale gases may comprise methane (CH₄) and one or more additional components, such as, ethane (C₂H₆), propane (C₃H₈), nitrogen (N₂), and combinations thereof. In various instances, exemplary shale gases may comprise various amounts of various constituents.

Exemplary shale gases may comprise methane (CH₄) at 28 mole % (mol %) to 94 mol %. In various instances, exemplary shale gases may comprise methane (CH₄) at 30 mol % to 94 mol %; 35 mol % to 94 mol %; 40 mol % to 90 mol %; 45 mol % to 85 mol %; 50 mol % to 80 mol %; 55 mol % to 75 mol %; or 60 mol % to 70 mol %. In various instances, exemplary shale gases may comprise methane (CH₄) at no greater than 94 mol %; no greater than 90 mol %; no greater than 85 mol %; no greater than 80 mol %; no greater than 75 mol %; no greater than 70 mol %; no greater than 65 mol %; no greater than 60 mol %; no greater than 55 mol %; no greater than 50 mol %; no greater than 45 mol %; no greater than 40 mol %; no greater than 35 mol %; or no greater than 30 mol %. In various instances, exemplary shale gases may comprise methane (CH₄) at no less than 28 mol %; no less than 30 mol %; no less than 35 mol %; no less than 40 mol %; no less than 45 mol %; no less than 50 mol %; no less than 55 mol %; no less than 60 mol %; no less than 65 mol %; no less than 70 mol %; no less than 75 mol %; no less than 80 mol %; no less than 85 mol %; no less than 90 mol %; or no less than 93 mol %.

Exemplary shale gases may comprise ethane (C₂H₆), when present, at 3 mol % to 20 mol %. In various instances, exemplary shale gases may comprise ethane (C₂H₆) at 4 mol % to 20 mol %; 5 mol % to 20 mol %; 6 mol % to 19 mol %; 7 mol % to 18 mol %; 8 mol % to 17 mol %; 9 mol % to 16 mol %; 10 mol % to 15 mol %; 11 mol % to 14 mol %; or 12 mol % to 13 mol %. In various instances, exemplary shale gases may comprise ethane (C₂H₆), when present, at no greater than 20 mol %; no greater than 18 mol %; no greater than 15 mol %; no greater than 12 mol %; no greater than 10 mol %; no greater than 8 mol %; or no greater than 5 mol %. In various instances, exemplary shale gases may comprise ethane (C₂H₆), when present, at no less than 3 mol %; no less than 5 mol %; no less than 7 mol %; no less than 10 mol %; no less than 13 mol %; no less than 15 mol %; or no less than 17 mol %.

Exemplary shale gases may comprise propane (C₃H₈), when present, at 1 mol % to 11 mol %. In various instances, exemplary shale gases may comprise propane (C₃H₈) at 1 mol % to 10 mol %; 1 mol % to 9 mol %; 2 mol % to 8 mol %; 3 mol % to 7 mol %; or 4 mol % to 6 mol %. In various instances, exemplary shale gases may comprise propane (C₃H₈), when present, at no greater than 11 mol %; no greater than 10 mol %; no greater than 9 mol %; no greater than 8 mol %; no greater than 7 mol %; no greater than 6 mol %; no greater than 5 mol %; no greater than 4 mol %; no greater than 3 mol %; or no greater than 2 mol %. In various instances, exemplary shale gases may comprise propane (C₃H₈), when present, at no less than 1 mol %; no less than 2 mol %; no less than 3 mol %; no less than 4 mol %; no less than 5 mol %; no less than 6 mol %; no less than 7 mol %; no less 8 mol %; no less than 9 mol %; or no less than 10 mol %.

Exemplary shale gases may comprise nitrogen (N₂), when present, at 2 mol % to 67 mol %. In various instances, exemplary shale gases may comprise nitrogen (N₂), when present, at 2 mol % to 66 mol %; 5 mol % to 65 mol %; 10 mol % to 60 mol %; 15 mol % to 55 mol %; 20 mol % to 50 mol %; 25 mol % to 45 mol %; or 30 mol % to 40 mol %. In various instances, exemplary shale gases may comprise nitrogen (N₂), when present, at no greater than 67 mol %; no greater than 65 mol %; no greater than 60 mol %; no greater than 55 mol %; no greater than 50 mol %; no greater than 45 mol %; no greater than 40 mol %; no greater than 30 mol %; no greater than 25 mol %; no greater than 20 mol %; no greater than 15 mol %; no greater than 10 mol %; or no greater than 5 mol %. In various instances, exemplary shale gases may comprise nitrogen (N₂), when present, at no less than 2 mol %; no less than 5 mol %; no less than 10 mol %; no less than 15 mol %; no less than 20 mol %; no less than 25 mol %; no less than 30 mol %; no less than 35 mol %; no less than 40 mol %; no less than 45 mol %; no less than 50 mol %; no less than 55 mol %; or no less than 60 mol %.

In some instances, nitrogen (N₂) gas may be added to exemplary gas compositions comprising shale gas to provide exemplary gas compositions comprising shale gas and additional nitrogen (N₂) gas.

Exemplary gas compositions comprising shale gas and additional nitrogen (N₂) gas may comprise methane (CH₄) at 7 mol % to 61 mol %. In various instances, exemplary gas compositions may comprise methane (CH₄) at 10 mol % to 60 mol %; 13 mol % to 57 mol %; 15 mol % to 55 mol %; 17 mol % to 53 mol %; 20 mol % to 50 mol %; 23 mol % to 47 mol %; 25 mol % to 45 mol %; 27 mol % to 43 mol %; 30 mol % to 40 mol %; or 33 mol % to 37 mol %. In various instances, exemplary gas compositions comprising shale gas and additional nitrogen (N₂) gas may comprise methane (CH₄) at no greater than 61 mol %; no greater than 60 mol %; no greater than 55 mol %; no greater than 50 mol %; no greater than 45 mol %; no greater than 40 mol %; no greater than 35 mol %; no greater than 30 mol %; no greater than 25 mol %; no greater than 20 mol % no greater than 15 mol %; or no greater than 10 mol %. In various instances, gas compositions comprising shale gas and additional nitrogen (N₂) gas may comprise methane (CH₄) at no less than 7 mol %; no less than 10 mol %; no less than 15 mol %; no less than 20 mol %; no less than 25 mol %; no less than 30 mol %; no less than 35 mol %; no less than 40 mol %; no less than 45 mol %; no less than 50 mol %; or no less than 55 mol %.

Exemplary gas compositions comprising shale gas and additional nitrogen (N₂) gas may comprise nitrogen (N₂) at 8 mol % to 90 mol %. In various instances, exemplary gas compositions comprising shale gas and additional nitrogen (N₂) gas may comprise nitrogen (N₂) at 10 mol % to 90 mol %; 15 mol % to 85 mol %; 20 mol % to 80 mol %; 25 mol % to 75 mol %; 30 mol % to 70 mol %; 35 mol % to 65 mol %; or 40 mol % to 60 mol %. In various instances, exemplary gas compositions comprising shale gas and additional nitrogen (N₂) gas may comprise nitrogen (N₂) at no greater than 90 mol %; no greater than 80 mol %; no greater than 70 mol %; no greater than 60 mol %; no greater than 50 mol %; no greater than 40 mol %; no greater than 30 mol %; no greater than 20 mol %; or no greater than 15 mol %. In various instances, exemplary gas compositions comprising shale gas and additional nitrogen (N₂) gas may comprise nitrogen (N₂) at no less than 8 mol %; no less than 10 mol %; no less than 20 mol %; no less than 30 mol %; no less than 40 mol %; no less than 50 mol %; no less than 60 mol %; no less than 70 mol %; or no less than 80 mol %.

### B. Exemplary Products

Exemplary fluid products generated by exemplary systems and methods disclosed and contemplated herein may comprise gas products and liquid products. In some instances, reactor effluent may comprise 90% to 96% unreacted reactants, where a remainder comprises various products such as those example products discussed below.

### 1. Exemplary Nitrogen (N)-Containing Products

Exemplary products may comprise nitrogen (N)-containing products. Exemplary nitrogen (N)-containing products may comprise one or more nitrogen (N)-containing compounds. In various instances, exemplary nitrogen (N)-containing compounds may comprise a combination of nitrogen (N), carbon (C), and hydrogen (H) atoms or a combination of nitrogen (N) and hydrogen (H) atoms. Exemplary nitrogen (N)-containing products may include products of formula CₓH_{y}N_{z} where x is an integer from 1 to 22, y is an integer from 1 to 38, and z is an integer from 1 to 9. Exemplary nitrogen (N)-containing products may further include ammonia (NH₃), hydrogen cyanide (HCN), acetonitrile (C₂H₃N), cyanamide (CH₂N₂), vinylamine (C₂H₅N), and 2-prop anamaine (C₃H₉N).

### 2. Exemplary Alkene Products

In various instances, exemplary products may comprise alkene products. Exemplary alkene products may comprise one or more C₂₋₄alkenes. Exemplary C₂₋₄alkenes may include ethene, propene, 1-butene, isobutene, cis-2-butene, trans-2-butene, and 1,3-butadiene.

### III. EXEMPLARY SYSTEMS

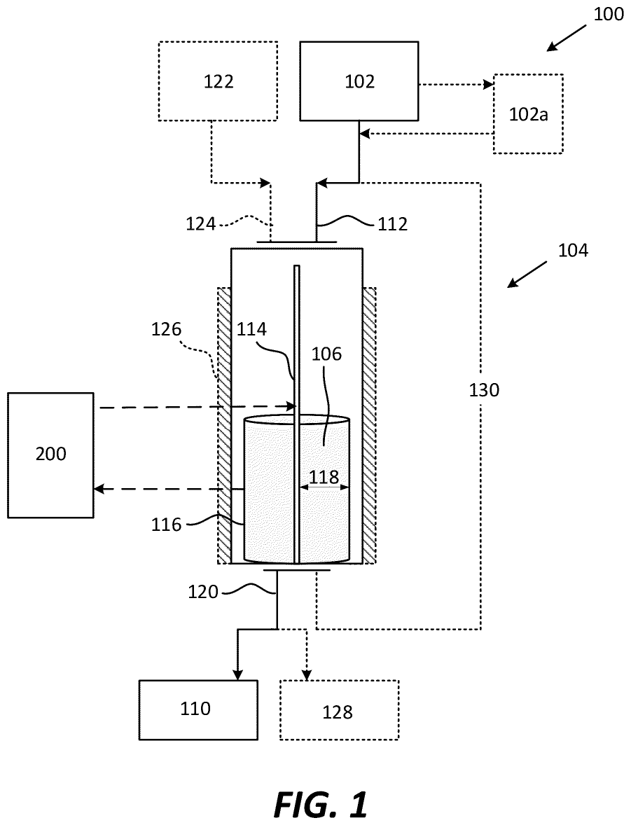

Various systems may be used to perform exemplary methods and techniques described herein. FIG. 1 is a schematic illustration of exemplary shale gas processing system 100. Broadly, shale gas processing system 100 is configured to process shale gas and generate fluid products. As shown, shale gas processing system 100 includes a shale gas source 102, a reactor 104, a voltage supply and monitor system 200, and a collection vessel 110. Various optional components are shown in dotted outline. Various electrical connections are shown in dashed lines. Other embodiments may include more or fewer components.

### A. Exemplary Reactors

Reactor 104 may be configured to receive and process shale gas from shale gas source 102. In the embodiment shown, reactor 104 is arranged vertically, such that material enters near a top portion of reactor 104, flows downward, and exits near a bottom portion of reactor 104.

Reactor 104 may include a first reactor inlet 112 configured to receive one or more fluids, such as shale gas, from a shale gas source 102. In some instances, reactor 104 may further include a second rector inlet 124 configured to receive one or more fluids, such as nitrogen (N₂) gas from a nitrogen (N₂) gas source 122. The first reactor inlet 112 and the second reactor inlet 124 may be positioned at an upper portion of the reactor 104.

The reactor 104 may include a “plasma zone” comprising an inner electrode 114, an outer electrode 116, and an inner volume defined between the inner electrode 114 and the outer electrode 116. In some instances, the plasma zone may extend through a portion of the interior of the reactor 104. In some instances, the plasma zone may extend through the interior of the reactor 104.

During typical operation, inner electrode 114 and outer electrode 116 cooperate to generate a plasma in the plasma zone. The plasma may be a non-thermal plasma. In some instances, the inner electrode 114 may extend through a portion of the interior of reactor 104. In some instances, the inner electrode 114 may extend through the entire interior of reactor 104. In some instances, the plasma zone may be cylindrical, wherein the outer electrode 116 annularly defines the exterior of the plasma zone.

Exemplary inner electrode 114 may comprise various materials. The particular material(s) for the inner electrode 114 may depend on the specific shale processing system. Exemplary materials for inner electrode 114, may include metals such as tungsten, nickel, and stainless steel.

Exemplary outer electrode 116 may comprise various materials. The particular material(s) for the outer electrode 116 may depend on the specific shale processing system.

Exemplary materials for outer electrode 116, may include metals such as stainless steel and copper. Inner electrode 114 and outer electrode 116 may be in electrical communication with a voltage supply and monitor system 200. In some instances, the voltage supply and monitor system 200 may be configured to provide a voltage to the inner electrode 114, thereby generating a plasma across a discharge gap 118.

A discharge gap 118 is defined as a distance between the inner electrode 114 and the outer electrode 116, where the distance is normal to both surfaces. A size of the discharge gap 118 may vary depending upon the specific implementation. The specific discharge gap size may influence the breakdown voltage of the plasma. Exemplary plasma zones may have a discharge gap of 0.1 mm to 150 mm. In some instances, exemplary plasma zones may have a discharge gap of 0.15 m to 150 mm; 0.25 mm to 125 mm; 0.50 mm to 100 mm; 1 mm to 99 mm; 2 mm to 98 mm; 5 mm to 95 mm; 8 mm to 92 mm; 10 mm to 90 mm; 12 mm to 88 mm; 15 mm to 85 mm; 18 mm to 82 mm; 20 mm to 80 mm; 22 mm to 78 mm; 25 mm to 75 mm; 28 mm to 72 mm; 30 mm to 70 mm; 32 mm to 68 mm; 35 mm to 65 mm; 38 mm to 62 mm; 40 mm to 60 mm; 42 mm to 58 mm; 45 mm to 55 mm; or 48 mm to 52 mm. In various instances, exemplary plasma zones may have a discharge gap of no greater than 150 mm; no greater than 125 mm; no greater than 100 mm; no greater than 90 mm; no greater than 80 mm; no greater than 75 mm; no greater than 70 mm; no greater than 60 mm; no greater than 50 mm; no greater than 40 mm; no greater than 30 mm; no greater than 25 mm; no greater than 20 mm; no greater than 15 mm; no greater than 10 mm; no greater than 5 mm; no greater than 1 mm; or no greater than 0.5 mm. In various instances, exemplary plasma zones may have a discharge gap of no less than 0.1 mm; no less than 0.25 mm; no less than 0.5 mm; no less than 1 mm; no less than 5 mm; no less than 10 mm; no less than 15 mm; no less than 20 mm; no less than 25 mm; no less than 30 mm; no less than 40 mm; no less than 50 mm; no less than 60 mm; no less than 70 mm; no less than 75 mm; no less than 80 mm; no less than 90 mm; no less than 100 mm; or no less than 125.

In some implementations, reactor 104 may further include a reactor temperature regulation unit 126. Exemplary reactor temperature regulation unit 126 may be arranged to maintain a predetermined temperature within the reactor. For instance, the reactor temperature regulation unit 126 may be configured to maintain a predetermined plasma zone temperature. The reactor temperature regulation unit 126 may be positioned at an exterior portion of the reactor 104. In various instances, exemplary reactor temperature regulation components 126 may maintain a plasma zone temperature of from 25° C. to 250° C.

Reactor 104 may further comprise a reactor outlet 120. Reactor outlet 120 may be positioned at a lower portion of the reactor. In various instances, the reactor outlet 120 may be configured to provide one or more fluid products, such as liquid products, to a collection vessel 110.

Exemplary collection vessels 110 may comprise various materials, such as stainless steel, carbon steel, glass, quartz, and combinations thereof. Exemplary collection vessels 110 may vary depending on the product's composition and volume. In various instances, exemplary collection vessels 110 may be air-free. For example, in some instances, exemplary collection vessels may be maintained under argon (Ar) or nitrogen (N₂) atmosphere.

In some instances, the reactor outlet 120 may be configured to provide one or more fluid products, such as liquid products, to an analysis unit 128 for product analysis. In various instances, the analysis unit 128 may be a mass spectrometer and/or a gas chromatograph (GC). Exemplary detectors for the gas chromatograph may comprise one or more of: a thermal conductivity detector (TCD), a flame ionization detector (FID), and a photoionization detector (PID). Specific configurations of analysis unit 128 may vary depending on the implementation.

In some instances, the reactor 104 may be configured to recycle unreacted gases from the reactor back to the first reactor inlet 112 via a fluid pathway 130.

In various instances, exemplary reactor 104 does not include catalyst material. Put another way, in various instances, exemplary reactor 104 may be catalyst material-free.

### B. Exemplary Shale Gas Sources

Exemplary shale gas source 102 may be any suitable reservoir containing shale gas. In various instances, shale gas source 102 may include various components configured to provide shale gas from a subterranean formation to the first reactor inlet. In some instances, the shale gas source 102 may be a shale gas well.

### C. Exemplary Pressure Regulation Components

In some implementations, exemplary system 100 may further include pressure regulation components, such as a depressurization unit 102a. Depressurization unit 102a may be in fluid communication with the shale gas source 102 and the first reactor inlet 112. The depressurization unit 102a may include various components arranged and configured to depressurize and provide shale gas from shale gas source 102 to the first reactor inlet 112. For instance, before the shale gas is provided to the first reactor inlet 112, exemplary depressurization unit 102a may decompress shale gas provided from shale gas source 102 to a pressure no greater than 0.3 Megapascal (MPa).

### D. Exemplary Voltage Supply and Monitor Systems

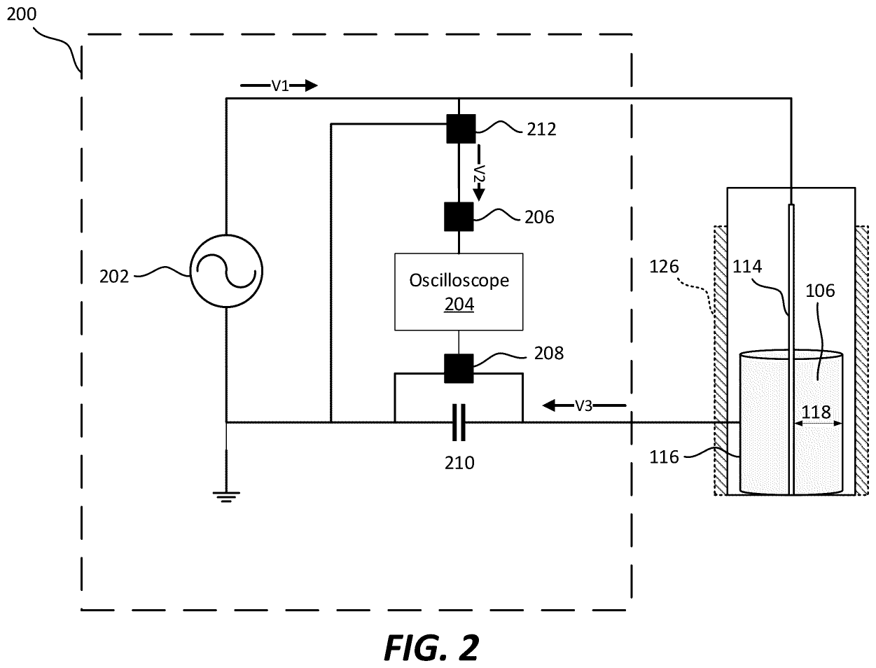

Voltage supply and monitor system 200 provides voltage to inner electrode 114 and monitors voltage from outer electrode 116. Various exemplary components of voltage supply and monitor system are shown in FIG. 2, discussed below.

As shown in FIG. 2, exemplary voltage supply and monitor system 200 may comprise a power source 202, an oscilloscope 204, probes 206,208, a capacitor 210, and a voltage attenuator 212. Other embodiments may include more or fewer components.

Exemplary power source 202 provides a voltage V1 to inner electrode 114. Typically, exemplary power source 202 is an alternating current (AC) power source. Exemplary power source 202 may be adjustable. In some instances, exemplary power source 202 may provide power intermittently, such as in pulses provided in a given time interval. In some instances, an exemplary interval may be every 1 second; every 2 seconds; every 5 seconds; every 10 seconds; every 15 seconds; every 20 seconds; every 30 seconds; every 45 seconds; every 60 seconds; every 90 seconds; every 120 seconds, or another interval.

In some implementations, the power source 202 is connected to a voltage attenuator 212. The voltage attenuator 212 decreases the first voltage V1 that is output from the power source 202. In various instances, the voltage attenuator 212 attenuates the voltage to a predetermined input:output ratio. As discussed in greater detail below, an exemplary input:output attenuation ratio may be 1000:1.

In some implementations, the oscilloscope 204 determines voltage waveforms with each probe 206, 208. The probes 206, 208 may extend from the oscilloscope 204. For example, a first probe 206 may extend from a first channel of the oscilloscope 204 and a second probe 208 may extend from a second channel of the oscilloscope 204. In some implementations, the second probe 208 is also connected to a grounding source. In some implementations, the first probe 206 is connected to the voltage attenuator 212 that attenuates the first voltage V1 that is output from the power source 202.

A second voltage V2 (i.e., the attenuated voltage) may be provided to the oscilloscope 204 by the first probe 206. In some instances, the second voltage V2 has the same frequency as the first voltage V1. In some instances, the voltage that is not output (i.e., the difference between the first voltage V1 and the second voltage V2) is sent to ground.

The oscilloscope 204 may measure a third voltage V3 across the capacitor 210 with the second probe 208. In some implementations, the capacitor 210 has a capacitance of 10 nano Farads (nF). In some instances, the capacitor's capacitance may be provided relative to the reactor's capacitance. In various instances, the ratio of the capacitor's capacitance to the reactor's capacitance may be 100:1 to 10,000:1. In some instances, the ratio of the capacitor's capacitance to the reactor's capacitance may be 100:1 to 9,000:1; 200:1 to 8,000:1; 300:1 to 7,000:1; 400:1 to 6,000:1; 500:1 to 5,000:1; 600:1 to 4,000:1; 700:1 to 3,000:1; to 800:1 to 2,000:1; or 900:1 to 1,000:1. In some instances, the ratio of the capacitor's capacitance to the reactor's capacitance may be no greater than 10,000:1; no greater than 9,000:1; no greater than 8,000:1; no greater than 7,000:1; no greater than 6,000:1; no greater than 5,000:1; no greater than 4,000:1; no greater than 3,000:1; no greater than 2,000:1; no greater than 1,000:1, no greater than 900:1; no greater than 800:1; no greater than 700:1; no greater than 600:1; no greater than 500:1; no greater than 400:1; no greater than 300:1; or no greater than 200:1. In some instances, the ratio of the capacitor's capacitance to the reactor's capacitance may be no less than 100:1; no less than 200:1; no less than 300:1; no less than 400:1; no less than 500:1; no less than 600:1; no less than 700:1; no less than 800:1; no less than 900:1; no less than 1,000:1; no less than 2,000:1; no less than 3,000:1; no less than 4,000:1; no less than 5,000:1; no less than 6,000:1; no less than 7,000:1; no less than 8,000:1; or no less than 9,000:1.

The third voltage V3 is voltage output from the outer electrode 116. In some implementations, the oscilloscope 204 determines a relationship between the second voltage V2 and the third voltage V3 to determine a power of a plasma that is generated in a plasma zone of the reactor.

### IV. EXEMPLARY METHODS

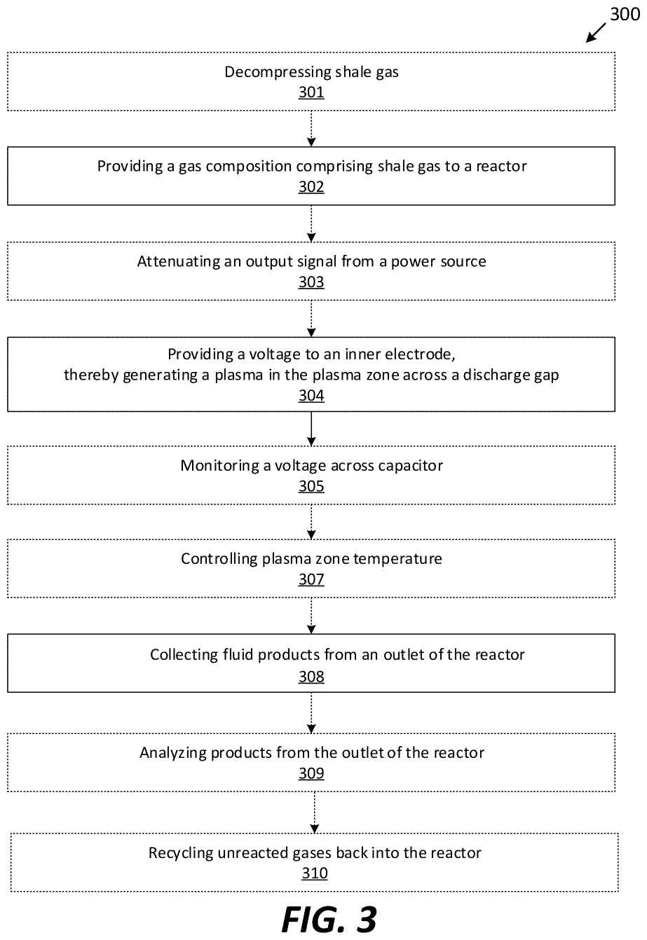

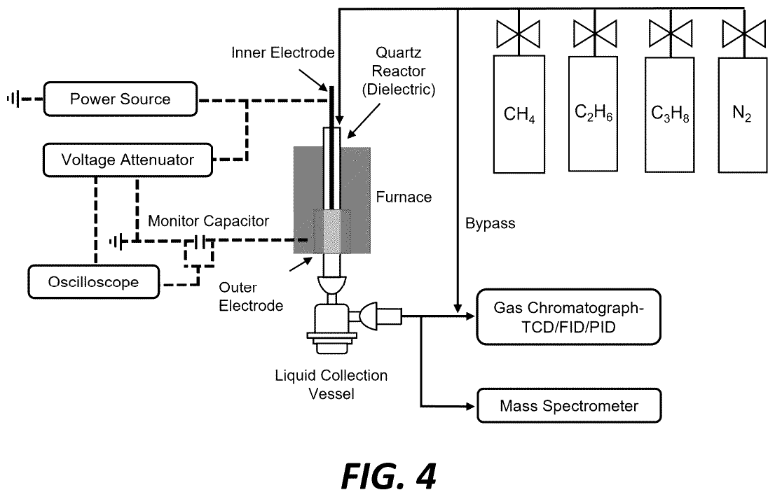

Exemplary methods for processing shale gases disclosed and contemplated herein include one or more operations. FIG. 3 is a schematic illustration of exemplary method 300 for processing shale gas. Broadly, method 300 includes providing a gas composition comprising shale gas to a reactor (operation 302), providing a voltage (e.g., first voltage V1) to an inner electrode disposed within a plasma zone of the reactor, thereby generating a plasma in the plasma zone across a discharge gap (operation 304), and collecting fluid products from an outlet of the reactor (operation 308). Other embodiments may include more or fewer operations.

### A. Providing Gas Composition to Reactor

In various instances, providing the gas composition to the reactor (operation 302) comprises providing shale gas at a first flowrate to a first inlet of the reactor.

In various instances, the first flowrate may be 5 standard cubic centimeters per minute (SCCM) (cm³/min) to 11,304 cm³/min. In some instances, the first flowrate is 10 cm³/min to 11,000 cm³/min; 20 cm³/min to 10,000 cm³/min; 30 cm³/min to 9,000 cm³/min; 40 cm³/min to 8,000 cm³/min; 50 cm³/min to 7,000 cm³/min; 60 cm³/min to 6,000 cm³/min ; 70 cm³/min to 5,000 cm³/min; 80 cm³/min to 4,000 cm³/min; 90 cm³/min to 3,000 cm³/min; 100 cm³/min to 2,000 cm³/min; 200 cm³/min to 1,000 cm³/min; 300 cm³/min to 900 cm³/min; 400 cm³/min to 800 cm³/min; or 500 cm³/min in to 700 cm³/min. In various instances, the first flowrate may be no greater than 11,304 cm³/min; no greater than 11,000 cm³/min; no greater than 10,000 cm³/min; no greater than 9,000 cm³/min; no greater than 8,000 cm³/min; no greater than 7,000 cm³/min; no greater than 6,000 cm³/min; no greater than 5,000 cm³/min; no greater than 4,000 cm³/min; no greater than 3,000 cm³/min; no greater than 2,000 cm³/min; no greater than 1,000 cm³/min; no greater than 900 cm³/min; no greater than 800 cm³/min; no greater than 700 cm³/min; no greater than 600 cm³/min; no greater than 500 cm³/min; no greater than 400 cm³/min.; no greater than 300 cm³/min; no greater than 200 cm³/min; no greater than 100 cm³/min; no greater than 90 cm³/min; no greater than 80 cm³/min; no greater than 70 cm³/min; no greater than 60 cm³/min; no greater than 50 cm³/min; no greater than 40 cm³/min; no greater than 30 cm³/min; no greater than 20 cm³/min; or no greater than 10 cm³/min. In various instances, the first flowrate may be no less than 5 cm³/min; no less than 10 cm³/min, no less than 20 cm³/min, no less than 30 cm³/min; no less than 40 cm³/min, no less than 50 cm³/min, no less than 60 cm³/min; no less than 70 cm³/min; no less than 80 cm³/min; no less than 90 cm³/min, no less than 100 cm³/min; no less than 200 cm³/min; no less than 300 cm³/min; no less than 400 cm³/min; no less than 500 cm³/min; no less than 600 cm³/min; no less than 700 cm³/min, no less than 800 cm³/min; no less than 900 cm³/min; no less than 1,000 cm³/min; no less than 2,000 cm³/min, no less than 3,000 cm³/min; no less than 4,000 cm³/min; no less than 5,000 cm³/min; no less than 6,000 cm³/min; no less than 7,000 cm³/min; no less than 8,000 cm³/min; no less than 9,000 cm³/min; no less than 10,000 cm³/min; or no less than 11,000 cm³/min.

In some instances, before providing the shale gas to the first inlet of the reactor, the shale gas may be decompressed to a pressure no greater than 0.3 Megapascal (MPa) (operation 301). In some instances, before providing the shale gas to the first inlet of the reactor, the shale gas may be decompressed to a pressure no greater than 0.25 MPa; no greater than 0.2 MPa; no greater than 0.15 MPa; or no greater than 0.1 MPa.

In some instances, providing the gas composition comprising shale gas to the reactor (operation 302) further comprises providing nitrogen (N₂) gas at a second flowrate to a second inlet of the reactor.

In various instances, the second flowrate may be 1 cm³/min to 10,174 cm³/min. In some instances, the second flowrate may be 5 cm³/min to 10,100 cm³/min; 10 cm³/min to 10,000 cm³/min; 20 cm³/min to 9,000 cm³/min; 30 cm³/min to 8,000 cm³/min; 40 cm³/min to 7,000 cm³/min; 50 cm³/min to 6,000 cm³/min; 60 cm³/min to 5,000 cm³/min; 70 cm³/min to 4,000 cm³/min; 80 cm³/min to 3,000 cm³/min; 90 cm³/min to 2,000 cm³/min; 100 cm³/min to 1,000 cm³/min; 200 cm³/min to 900 cm³/min; 300 cm³/min to 800 cm³/min; 400 cm³/min to 700 cm³/min; or 500 cm³/min to 600 cm³/min. In various instances, the second flowrate may be no greater than 10,174 cm³/min; no greater than 10,000 cm³/min; no greater than 9,000 cm³/min; no greater than 8,000 cm³/min; no greater than 7,000 cm³/min; no greater than 6,000 cm³/min; no greater than 5,000 cm³/min; no greater than 4,000 cm³/min; no greater than 3,000 cm³/min; no greater than 2,000 cm³/min; no greater than 1,000 cm³/min; no greater than 900 cm³/min; no greater than 800 cm³/min; no greater than 700 cm³/min; no greater than 600 cm³/min; no greater than 500 cm³/min; no greater than 400 cm³/min; no greater than 300 cm³/min; no greater than 200 cm³/min; no greater than 100 cm³/min; no greater than 90 cm³/min; no greater than 80 cm³/min; no greater than 70 cm³/min; no greater than 60 cm³/min; no greater than 50 cm³/min; no greater than 40 cm³/min; no greater than 30 cm³/min; no greater than 20 cm³/min; or no greater than 10 cm³/min. In various instances, the second flowrate may be no less than 4 cm³/min; no less than 10 cm³/min; no less than 20 cm³/min no less than 30 cm³/min; no less than 40 cm³/min; no less than 50 cm³/min; no less than 60 cm³/min; no less than 70 cm³/min no less than 80 cm³/min; no less than 90 cm³/min; no less than 100 cm³/min; no less than 200 cm³/min; no less than 300 cm³/min; no less than 400 cm³/min; no less than 500 cm³/min; no less than 600 cm³/min; no less than 700 cm³/min, no less than 800 cm³/min; no less than 900 cm³/min; no less than 1,000 cm³/min; no less than 2,000 cm³/min; no less than 3,000 cm³/min; no less than 4,000 cm³/min; no less than 5,000 cm³/min; no less than 6,000 cm³/min; no less than 7,000 cm³/min, no less than 8,000 cm³/min; no less than 9,000 cm³/min; or no less than 10,000 cm³/min.

### B. Providing a Voltage to Inner Electrode

After providing the gas composition comprising shale gas to the reactor (operation 302), exemplary method 300 may further comprise providing a voltage (e.g., first voltage V1) to an inner electrode disposed within a plasma zone of the reactor, thereby generating a plasma in the plasma zone across a discharge gap (operation 304). The plasma generated in the plasma zone may be a non-thermal plasma. Exemplary voltages may be provided from a power source, such as power source 202.

In various instances, the voltage provided to the inner electrode may be 6 kV to 9 kV. In some instances, the voltage provided to the inner electrode may be 6.5 kV to 8.75 kV; 7.0 kV to 8.7 kV; 7.5 kV to 8.5 kV; 7.6 kV to 8.4 kV; 7.7 kV to 8.3 kV; 7.8 kV to 8.2 kV; or 7.9 kV to 8.1 kV. In various instances, the voltage provided to the inner electrode may be no greater than no greater than 9 kV; no greater than 8.75 kV; no greater than 8.7 kV; 8.5 kV; no greater than 8.4 kV; no greater than 8.3 kV; no greater than 8.2 kV; no greater than 8.1 kV; no greater than 8.0 kV; no greater than 7.9 kV; no greater than 7.8 kV; no greater than 7.7 kV; or no greater than 7.6 kV. In various instances, the voltage provided to the inner electrode may be no less than 6; no less than 6.5 kV; no less than 7.0 kV; no less than 7.5 kV; no less than 7.6 kV; no less than 7.7 kV; no less than 7.8 kV; no less than 7.9 kV; no less than 8.0 kV; no less than 8.1 kV; no less than 8.2 kV; no less than 8.3 kV; or no less than 8.4 kV.

In various instances, the voltage may be provided to the inner electrode at a frequency of 2 kHz to 700 kHz. In some instances, the voltage may be provided to the inner electrode at a frequency of 5 kHz to 675 kHz; 10 kHz to 650 kHz; 15 kHz to 625 kHz; 20 kHz to 600 kHz; 25 kHz to 575 kHz; 30 kHz to 550 kHz; 35 kHz to 525 kHz; 40 kHz to 500 kHz; 45 kHz to 475 kHz; 50 kHz to 450 kHz; 55 kHz to 425 kHz; 60 kHz to 400 kHz; 65 kHz to 375 kHz; 70 kHz to 350 kHz; 75 kHz to 325 kHz; 80 kHz to 300 kHz; 85 kHz to 275 kHz; 90 kHz to 250 kHz; 95 kHz to 225 kHz; 100 kHz to 200 kHz; or 125 kHz to 175 kHz. In some instances, the voltage may be provided to the inner electrode at a frequency of no greater than 700 kHz; no greater than 650 Hz; no greater than 600 kHz; no greater than 550 kHz; no greater than 500 kHz; no greater than 450 kHz; no greater than 400 kHz; no greater than 350 kHz; no greater than 300 kHz; no greater than 250 kHz; no greater than 200 kHz; no greater than 150 kHz; no greater than 100 kHz; no greater than 50 kHz; no greater than 25 kHz; no greater than 20 kHz; no greater than 15 kHz; no greater than 10 kHz; or no greater than 5 kHz. In some instances, the voltage may be provided to the inner electrode at a frequency of no less than 5 kHz; no less than 10 kHz; no less than 15 kHz; no less than 20 kHz; no less than 25 kHz; no less than 50 kHz; no less than 100 kHz; no less than 150 kHz; no less than 200 kHz; no less than 250 kHz; no less than 300 kHz; no less than 350 kHz; no less than 400 kHz; no less than 450 kHz; no less than 500 kHz; no less than 550 kHz; no less than 600 kHz; or no less than 650 kHz.

In some instances, before providing the voltage to the inner electrode (operation 304), method 300 may comprise attenuating an output signal from the power source (operation 303). For example, in some instances the output signal may be attenuated to an input:output ratio of 1000:1.

In some instances, while providing the voltage to the inner electrode, the voltage across a capacitor may be monitored (operation 305). In various instances, the voltage across the capacitor may be monitored continuously with a probe, such as probe 208.

In various instances, the specific energy input (SEI) to the plasma zone can be determined. Specific energy input (SEI) is the ratio of the calculated plasma power to the gas flow rate. As described above, in various instances, an oscilloscope (e.g., oscilloscope 204) may calculate the power of the plasma that is generated in a plasma zone from a relationship between the second voltage V2 (e.g., attenuated input voltage V2, measured with probe 206) and the third voltage V3 (e.g., output voltage V3 from the outer electrode 116, measured across the capacitor 210 with the second probe 208).

In various instances, the specific energy input may be from 1 kJ/L to 100 kJ/L. In some instances, the specific energy input may be from 1 kJ/L to 99 kJ/L; 5 kJ/L to 95 kJ/L; 10 kJ/L to 90 kJ/L; 15 kJ/L to 85 kJ/L; 20 kJ/L to 80 kJ/L; 25 kJ/L to 75 kJ/L; 30 kJ to 70 kJ/L; 35 kJ/L to 65 kJ/L; 40 kJ/L to 60 kJ/L; 9 kJ/L to 15 kJ/L; or 45 kJ/L to 55 kJ/L. In some instances, the specific energy input may be from no greater than 100 kJ/L; no greater than 90 kJ/L; no greater than 80 kJ/L; no greater than 70 kJ/L; no greater than 60 kJ/L; no greater than 50 kJ/L; no greater than 40 kJ/L; no greater than 35 kJ/L; no greater than 30 kJ/L; no greater than 25 kJ/L; no greater than 20 kJ/L; no greater than 15 kJ/L; no greater than 10 kJ/L; or no greater than 5 kJ/L. In some instances, the specific energy input may be from no less than 5 kJ/L; no less than 10 kJ/L; no less than 15 kJ/L; no less than 20 kJ/L; no less than 25 kJ/L; no less than 30 kJ/L; no less than 40 kJ/L; no less than 50 kJ/L; no less than 60 kJ/L; no less than 70 kJ/L; no less than 80 kJ/L; no less than 90 kJ/L; or no less than 95 kJ/L.

### C. Controlling Plasma Zone Temperature

In some implementations, while providing the voltage to the inner electrode (operation 304), the plasma zone's temperature may be controlled using a temperature controlling device (operation 307). In various instances, the plasma zone may be maintained at a temperature of 25° C. to 250° C. In various instances, the plasma zone may be maintained at a temperature of 30° C. to 250° C.; 40° C. to 240° C.; 50° C. to 230° C.; 60° C. to 220° C.; 70° C. to 210° C.; 80° C. to 200° C.; 90° C. to 190° C.; 100° C. to 180° C.; 110° C. to 170° C.; 120° C. to 160° C.; or 130° C. to 150° C. In various instances, the plasma zone may be maintained at a temperature of no greater than 250° C.; no greater than 225° C.; no greater than 200° C.; no greater than 175° C.; no greater than 150° C.; no greater than 125° C.; no greater than 100° C.; no greater than 75° C.; no greater than 50° C.; or no greater than 30° C. In various instances, the plasma zone may be maintained at a temperature of no less than 25° C.; no less than 30° C.; no less than 50° C.; no less than 75° C.; no less than 100° C.; no less than 125° C.; no less than 150° C.; no less than 175° C.; no less than 200° C.; no less than 225° C.; or no less than 240° C.

### D. Collecting Products and Recycling Unreacted Gases

In various instances, while providing the voltage to the inner electrode (operation 304), exemplary method 300 may further comprise collecting fluid products from the reactor outlet (operation 308). In some instances, collecting fluid products (operation 308) may include cooling fluid products from the reactor outlet to generate liquid products.

In some instances, exemplary method 300 may comprise sending fluid products from the reactor outlet to an analysis unit, such as analysis unit 128, where the fluid products may be analyzed (operation 309). In various instances, exemplary analysis unit 128 may provide output data regarding reactant conversion, product types, and product selectivity. In response to the output data from analysis unit 128, various process inputs, such as flow rate, power, compositions, and temperature may be adjusted. Such adjustments in response to output data from analysis unit 128, may improve the processes' reactant conversions and production rates.

In some instances, exemplary method 300 may further comprise recycling unreacted gases back to the first inlet of the reactor (operation 310).

### V. EXPERIMENTAL EXAMPLES

Without limiting the scope of the instant disclosure, experimental examples of embodiments discussed above were prepared and the results are discussed below.

The following examples demonstrate that using a variety of feed gas compositions and plasma power inputs, a wide range of products, both gas and liquid, may be produced, the products comprising carbon-carbon (C-C), carbon-nitrogen (C—N), and nitrogen-hydrogen (N-H) bonds.

### Example 1

An A/C power source was used to supply an input power of 10 W to a dielectric barrier discharge reactor. The reactor had a discharge gap of 1.75 mm between a tungsten inner electrode and stainless-steel outer electrode. A constant temperature of200° C. was maintained by an external furnace. A feed gas composition flowrate of 50 mL/min was controlled by mass flow controllers, and the feed gas composition was 61 mol % CH₄, 20 mol % C₂H₆, 11 mol % C₃H₈, and 8 mol % N₂. The gas phase reactor effluent was continually monitored by an in-line mass spectrometer and an in-line gas chromatographer equipped with a thermal conductivity detector (TCD), flame ionization detector (FID), and photoionization detector (PID). The conversion of the reactants was calculated using external calibration curves. For each reactant, the reactant conversion was calculated as the moles of the reactant converted to products divided by the moles of a reactant in the feed gas compositions. The initial hydrocarbon reactant conversions were 4.6%, 11.6%, and 17.2% for methane, ethane, and propane, respectively (Table 1). No N₂ conversion was measured for this reaction.

TABLE 1
Reactant conversion (%) with standard deviation (%) for the reaction
with a feed gas composition of 61 mol % CH₄, 20 mol % C₂H₆,
11 mol % C₃H₈, and 8 mol % N₂.

| | Conversion (%) | Std. Dev. (%) |
|---|---|---|
| Methane | 4.6 | 0.09 |
| Ethane | 11.6 | 0.4 |
| Propane | 17.2 | 0.4 |
| Nitrogen | 0 | 0 |

The gas phase hydrocarbon product selectivity was calculated using FID peak areas and the carbon number of each product, shown in Equation 1.

$$S ( \% ) = \frac{\frac{area desired product}{carbon number desired product}}{Σ \frac{area product}{carbon number product}} \times 100 \qquad ( 1 )$$

Table 2 shows the initial gas phase carbon product selectivity to ethylene and acetylene are 40.8% and 18.8%, respectively, corresponding to production rates of 0.22 and 0.10 μmol/mL/s.

TABLE 2
Product selectivity (%) with standard deviation (%)
for the reaction with a feed gas composition of 61 mol
% CH₄, 20 mol % C₂H₆, 11 mol % C₃H₈, and 8 mol % N₂.

| | Selectivity (%) | Std. Dev. (%) |
|---|---|---|
| Ethylene | 40.8 | 0.04 |
| Acetylene | 18.8 | 0.4 |
| Propylene | 11.9 | 0.04 |
| n-Butane | 9.9 | 0.6 |
| 1-Butene | 0.7 | 0.03 |
| Isobutene | 0.6 | 0.09 |
| 1,3-Butadiene | 0.4 | 0.1 |
| cis/trans-2-Butene | 0.4 | 0.1 |
| C5 | 3.1 | 0.4 |
| C6 | 0.8 | 0.002 |
| C7 | 0.3 | 0.04 |
| Unknown | 12.3 | 1.0 |

The reaction was initially 73.6% selective towards alkenes and alkynes in the gas phase. Under these conditions, the majority of products were gas phase hydrocarbons, and the carbon balance was determined to be 98%. The carbon balance was determined by calculating the sum of the moles of carbon unreacted from the feed gas composition and the moles of carbon in the gas phase products from the reaction, and dividing this sum by the total moles of carbon fed to the reactor.

After 5-hours of reaction time, the liquid was collected using dichloromethane (CH₂Cl₂). The solvent was evaporated overnight, and the weight of the remaining liquid product was determined to be 0.50±0.42 mg, or a production rate of 0.026±0.022 μg/mL/s. ¹-H nuclear magnetic resonance (NMR) was used to determine that the CH₂Cl₂ had evaporated and 99.75% of the weight was due to the liquid product.

### Example 2

The experimental conditions of Example 1 were maintained (10 W plasma, 200° C., 50 mL/min), and the feed gas composition was changed to 35 mol % CH₄, 10 mol % C₂H₆, 5 mol % C₃H₈, and 50 mol % N₂. Here the initial conversions were 5.2%, 15%, 32.1% and 2.5% for methane, ethane, propane, and nitrogen, respectively (Table 3).

TABLE 3
Reactant conversion (%) with standard deviation (%)
for the reaction with a feed gas composition of 35 mol
% CH₄, 10 mol % C₂H₆, 5 mol % C₃H₈, and 50 mol % N₂.

| | Conversion (%) | Std. Dev. (%) |
|---|---|---|
| Methane | 5.2 | 0.2 |
| Ethane | 15.0 | 0.2 |
| Propane | 32.1 | 0.7 |
| Nitrogen | 2.5 | 0.1 |

As shown in Table 4, the initial gas phase carbon product selectivity to ethylene and acetylene are 32.8% and 22.1%, respectively, corresponded to production rates of 0.16 and 0.11 μmol/mL/s.

TABLE 4
Product selectivity (%) with standard deviation (%)
for the reaction with a feed gas composition of 35 mol
% CH₄, 10 mol % C₂H₆, 5 mol % C₃H₈, and 50 mol % N₂.

| | Selectivity (%) | Std. Dev. (%) |
|---|---|---|
| Ethylene | 32.8 | 3.8 |
| Acetylene | 22.1 | 2.5 |
| Propylene | 10.0 | 1.1 |
| n-Butane | 12.8 | 1.6 |
| 1-Butene | 0.7 | 0.04 |
| Isobutene | 0.9 | 0.01 |
| 1,3-Butadiene | 0.2 | 0.05 |
| cis/trans-2-Butene | 0.5 | 0.05 |
| C5 | 3.4 | 0.2 |
| C6 | 0.8 | 0.03 |
| C7 | 0.2 | 0.02 |
| Unknown | 15.6 | 0.5 |

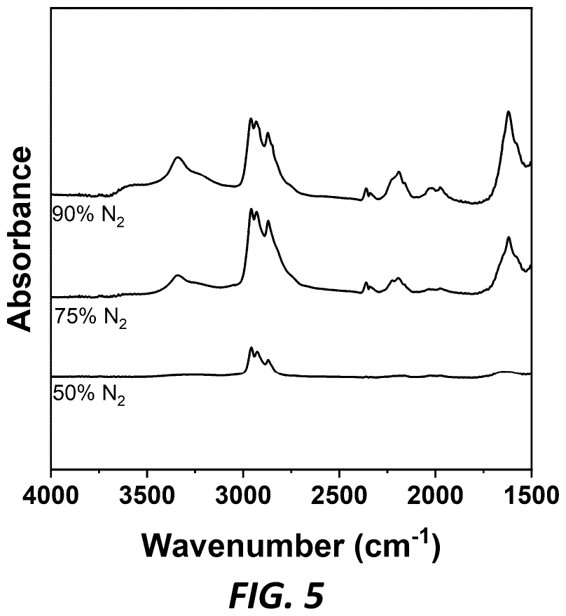

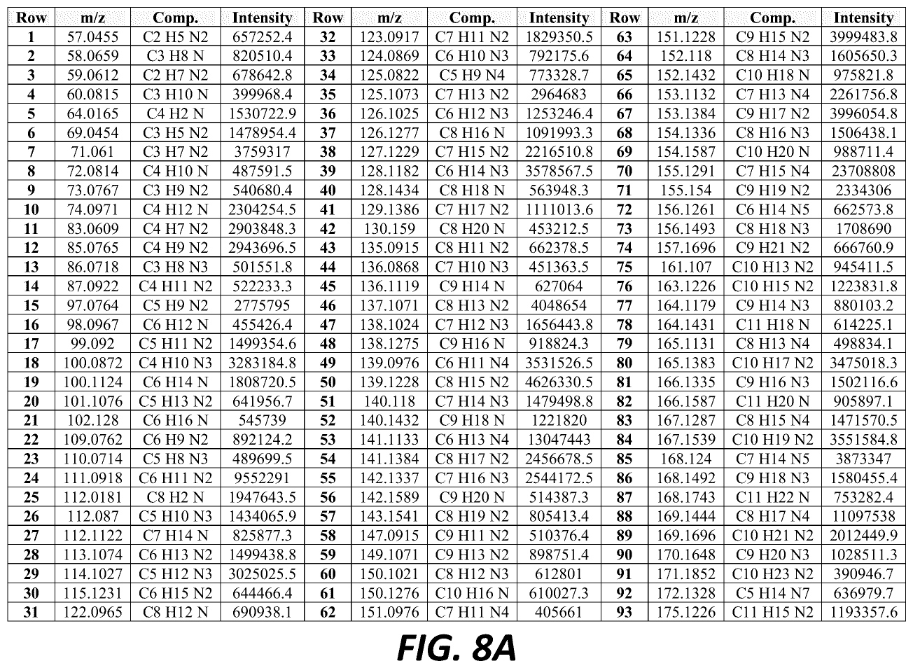

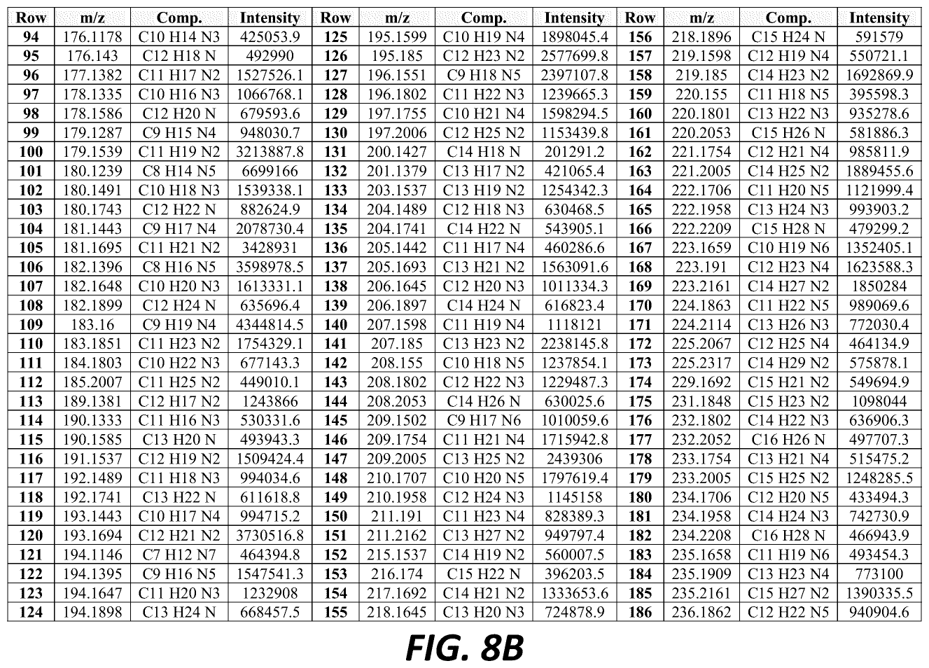

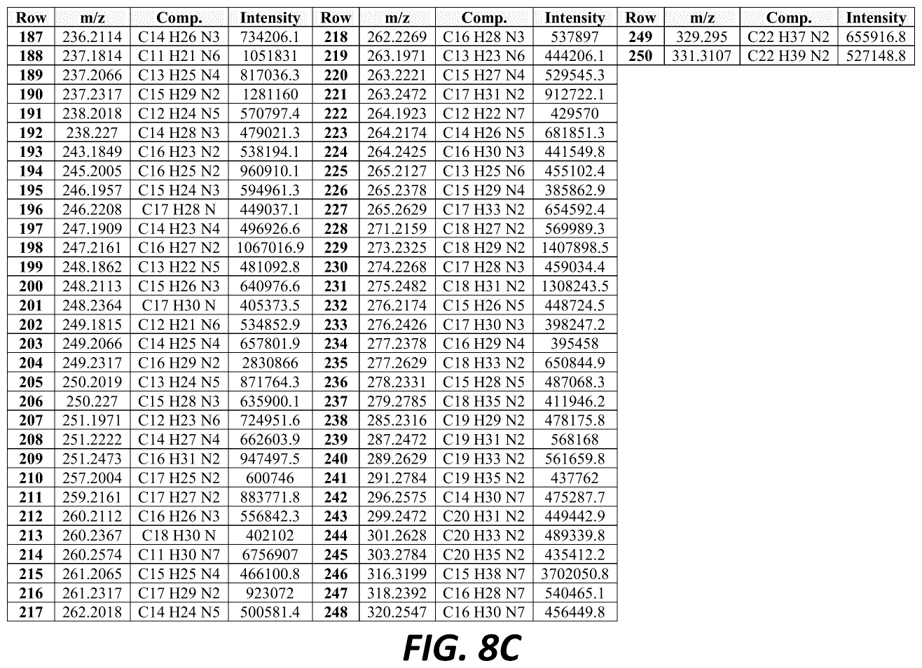

The initial selectivity to alkene and alkynes in the gas phase hydrocarbons was 67.2% at this condition. The gas phase carbon product selectivities shifted to higher molecular weight products (C₄₊) compared to Example 1. The carbon balance was determined to be 97%. In-line mass spectrometry assisted in the identification of additional products such as hydrogen cyanide, acetonitrile, cyanamide, vinylamine, and 2-propanamaine. At this condition, 2.5 nmol/mL/s of ammonia was produced, calculated by an external calibration curve. The liquid production rate increased to 0.17±0.07 μg/mL/s for a 5-hour reaction. Attenuated total reflectance infrared spectroscopy (ATR-IR) of the liquid product showed small features in the amine and nitrile regions (FIG. 5). High resolution electrospray ionization mass spectrometry (ESI-MS) of the liquid product was used to determine the chemical formulas (CₓH_{y}N_{z}) of the 250 compounds with the highest intensity in the spectra (FIGS. 8A-8C), where repeating units of —H₂—, —CH₂— and —HCN— were observed.

### Example 3

The experimental conditions of Example 1 were maintained (10 W plasma, 200° C., 50 mL/min), and the feed gas composition was changed to 17 mol % CH₄, 5 mol % C₂H₆, 3 mol % C₃H₈, and 75 mol % N₂. Here the initial conversions were 8.5%, 25.0%, 33.9%, and 2.5% for methane, ethane, propane, and nitrogen, respectively (Table 5).

TABLE 5
Reactant conversion with standard deviation for the
reaction with a feed gas composition of 17 mol % CH₄, 5 mol % C₂H₆,
3 mol % C₃H₈, and 75 mol % N₂.

| | Conversion (%) | Std. Dev. (%) |
|---|---|---|
| Methane | 8.6 | 1.1 |
| Ethane | 25.0 | 1.7 |
| Propane | 33.9 | 6.0 |
| Nitrogen | 2.5 | 0.2 |

As shown in Table 6, the initial gas phase carbon product selectivity to ethylene and acetylene are 28.6% and 23.9%, respectively, corresponded to production rates of 0.091 and 0.076 μmol/mL/s.

TABLE 6
Product selectivity (%) with standard deviation (%)
for the reaction with a feed gas composition of 17 mol
% CH₄, 5 mol % C₂H₆, 3 mol % C₃H₈, and 75 mol % N₂.

| | Selectivity (%) | Std. Dev. (%) |
|---|---|---|
| Ethylene | 28.6 | 1.0 |
| Acetylene | 23.9 | 0.6 |
| Propylene | 8.6 | 0.01 |
| n-Butane | 12.6 | 0.3 |
| 1-Butene | 0.8 | 0.05 |
| Isobutene | 1.5 | 0.1 |
| 1,3-Butadiene | 0.3 | 0.04 |
| cis/trans-2-Butene | 0.8 | 0.09 |
| C5 | 4.1 | 0.3 |
| C6 | 1.2 | 0.4 |
| C7 | 0.6 | 0.2 |
| Unknown | 17 | 1.2 |

The reaction was initially 64.5% selective towards alkenes and alkynes in the gas phase. The in-line mass spectrometer identified additional products such as hydrogen cyanide, acetonitrile, cyanamide, vinylamine, and 2-propanamaine. At this condition, 3.6 nmol/mL/s NH₃ was produced, and the liquid production rate increased to 1.10±0.29 μg/mL/s for a 5-hour reaction with a carbon balance of 93%. Here, the carbon balance was determined by calculating the sum of the moles of carbon unreacted from the feed gas composition, the moles of carbon in the gas phase products from the reaction, and the moles of carbon in the liquid determined by elemental analysis, and dividing this sum by the total moles of carbon fed to the reactor. The N/C ratio of the liquid product was 0.38±0.01. Attenuated total reflectance infrared spectroscopy (ATR-IR) of the liquid product shows features in the amine and nitrile regions (FIG. 5).

### Example 4

The experimental conditions of Example 1 were maintained (10 W plasma, 200° C., 50 mL/min), and the feed gas composition was changed to 7 mol % CH₄, 2 mol % C₂H₆, 1 mol % and 90 mol % N₂. Here the initial conversions were 14.8%, 37.9%, 45.7% and 2.7% for methane, ethane, propane, and nitrogen, respectively (Table 7).

TABLE 7
Reactant conversion (%) with standard deviation (%)
for the reaction with a feed gas composition of 7 mol
% CH₄, 2 mol % C₂H₆, 1 mol % C₃H₈, and 90 mol % N₂.

| | Conversion (%) | Std. Dev. (%) |
|---|---|---|
| Methane | 14.8 | 4.0 |
| Ethane | 37.9 | 1.1 |
| Propane | 45.7 | 7.0 |
| Nitrogen | 2.7 | 0.1 |

As shown in Table 8, the initial gas phase carbon product selectivity to ethylene and acetylene are 31.8% and 23.5%, respectively, corresponding to production rates of 0.049 and 0.036 μmol/mL/s.

TABLE 8
Product selectivity (%) with standard deviation (%)
for the reaction with a feed gas composition of 7 mol
% CH₄, 2 mol % C₂H₆, 1 mol % C₃H₈, and 90 mol % N₂.

| | Selectivity (%) | Std. Dev. (%) |
|---|---|---|
| Ethylene | 31.8 | 3.2 |
| Acetylene | 23.5 | 1.0 |
| Propylene | 8.5 | 0.6 |
| n-Butane | 10.4 | 1.2 |
| 1-Butene | 0.9 | 0.004 |
| Isobutene | 1.9 | 0.3 |
| 1,3-Butadiene | 0.5 | 0.2 |
| cis/trans-2-Butene | 0.9 | 0.06 |
| C5 | 4.0 | 0.5 |
| C6 | 0.9 | 0.3 |
| C7 | 0.7 | 0.05 |
| Unknown | 16.0 | 1.1 |

The reaction was initially 68% selective towards alkenes and alkynes in the gas phase. The in-line mass spectrometer identified additional products such as hydrogen cyanide, acetonitrile, cyanamide, vinylamine, and 2-propanamaine.

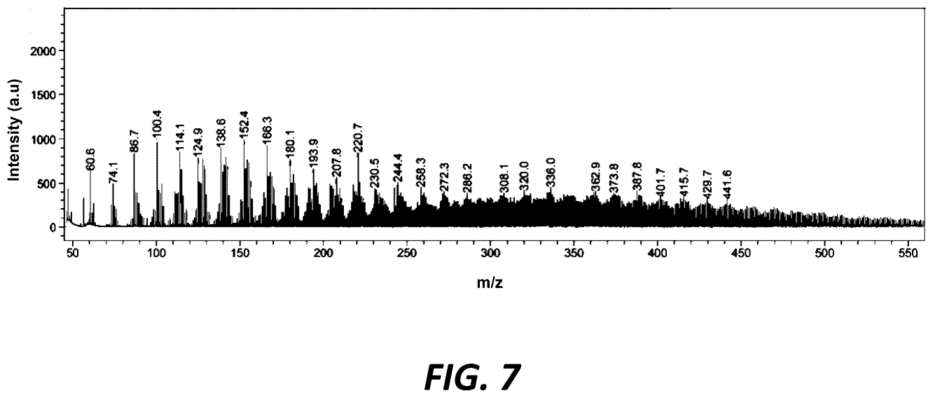

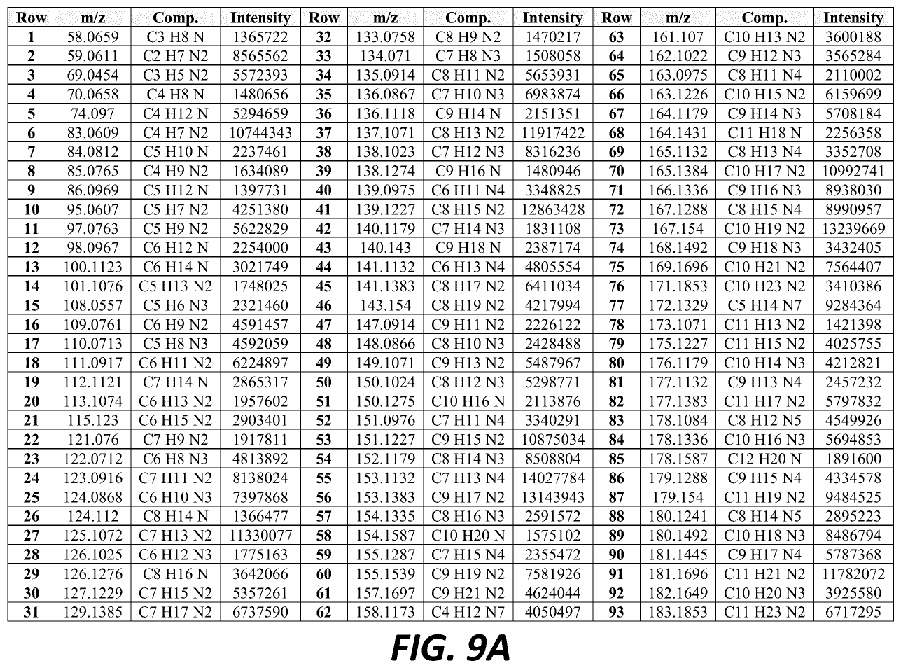

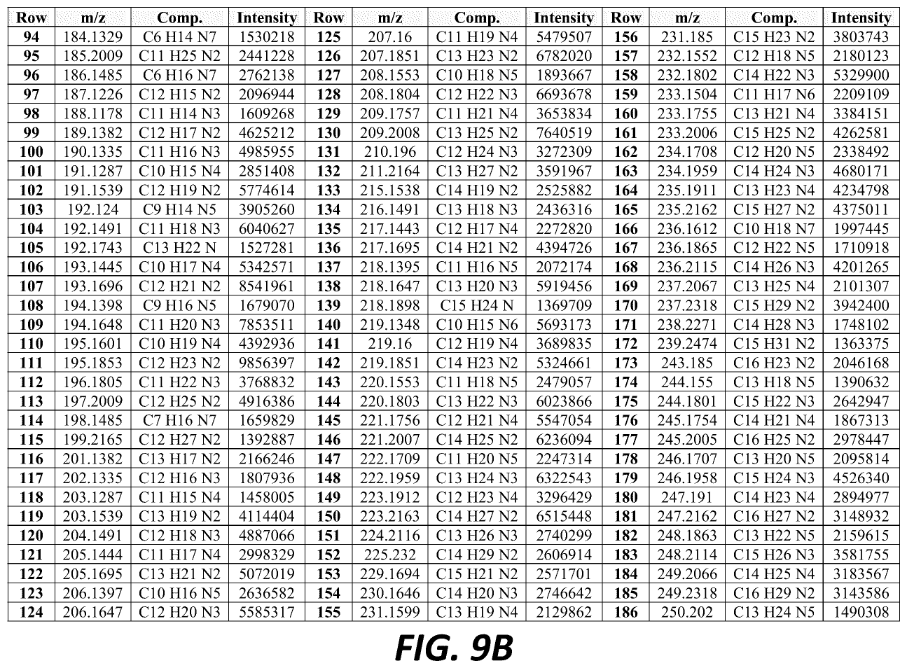

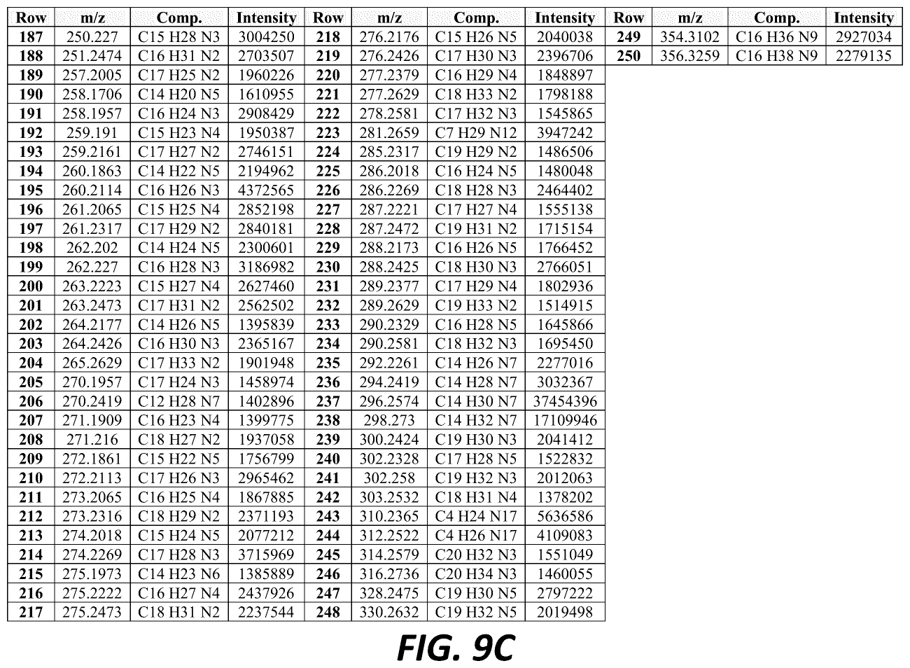

At this condition, 5.0 nmol/mL/s of NH₃ was produced, and the liquid production rate increased to 1.4±0.20 μg/mL/s for a 5-hour reaction with a carbon balance of 80%. The lower carbon balance at this condition indicates that a portion of the liquid product is volatile and not able to be collected. Elemental analysis of the liquid product provided an N/C ratio of 0.33±0.03. Attenuated total reflectance infrared spectroscopy (ATR-IR) of the liquid product showed features in the amine and nitrile regions (FIG. 5). High resolution electrospray ionization mass spectrometry (ESI-MS) of the liquid product was used to determine the chemical formulas (CₓH_{y}N_{z}) of the 250 compounds with the highest intensity in the spectra (FIGS. 9A-9C). Repeating units of —H₂—, —CH₂— and —HCN— were observed. Matrix assisted laser desorption/ionization-time of flight mass spectrometry (MALDI-TOF-MS) showed that the liquid is a mixture of compounds with masses up to ˜750 g/mol (FIG. 7).

### Example 5

The experimental conditions of Example 1 were maintained (10 W plasma, 200° C., 50 mL/min), and the feed gas composition was changed to 94 mol % CH₄, 3 mol % C₂H₆, 1 mol % C₃H₈, and 2 mol % N₂. Here the initial conversion for methane was 7.8% (Table 9).

TABLE 9
Reactant conversion (%) with standard deviation (%)
for the reaction with a feed gas composition of 94 mol
% CH₄, 3 mol % C₂H₆, 1 mol % C₃H₈, and 2 mol % N₂.

| | Conversion (%) | Std. Dev. (%) |
|---|---|---|
| Methane | 7.8 | 0.6 |
| Ethane | — | — |
| Propane | — | — |
| Nitrogen | — | — |

Ethane and propane conversion were not calculated as their peak areas on the GC increased during reaction, indicating that more of these gases were being formed by the reaction than consumed. No N₂ conversion was measured for this reaction. As shown in Table 10, the initial gas phase carbon product selectivity to ethylene and acetylene are 28.9% and 27.0%, respectively, corresponding to production rates of 0.089 and 0.082 μmol/mL/s.

TABLE 10
Product selectivity (%) with standard deviation (%)
for the reaction with a feed gas composition of 94 mol
% CH₄, 3 mol % C₂H₆, 1 mol % C₃H₈, and 2 mol % N₂.

| | Selectivity (%) | Std. Dev. (%) |
|---|---|---|
| Ethylene | 28.9 | 0.8 |
| Acetylene | 27.0 | 1.9 |
| Propylene | 7.2 | 0.7 |
| n-Butane | 8.7 | 0.04 |
| 1-Butene | 1.0 | 0.2 |
| Isobutene | 1.4 | 0.3 |
| 1,3-Butadiene | 0.5 | 0.06 |
| cis/trans-2-Butene | 0.8 | 0.04 |
| C5 | 4.0 | 0.6 |
| C6 | 1.1 | 0.1 |
| C7 | 0.7 | 0.4 |
| Unknown | 18.7 | 1.2 |

The reaction was initially 66.8% selective towards alkenes and alkynes in the gas phase. The carbon balance was calculated to be 99%. At this condition, the liquid production rate was 0.02 μg/mL/s for a 5-hour reaction.

### Example 6

The experimental conditions of Example 1 were maintained (10 W plasma, 200° C., 50 mL/min), and the feed gas composition was changed to 28 mol % CH_{4 , 4} mol % C₂H₆, 1 mol % C₃H₈, and 67 mol % N₂. Here the initial conversions were 11.7%, 18.6%, and 1.9% for methane, ethane, and nitrogen, respectively (Table 11).

TABLE 11
Reactant conversion (%) with standard deviation (%)
for the reaction with a feed gas composition of 28 mol
% CH₄, 4 mol % C₂H₆, 1 mol % C₃H₈, and 67 mol % N₂.

| | Conversion % | Std. Dev. (%) |
|---|---|---|
| Methane | 11.7 | 0.5 |
| Ethane | 18.6 | 0.1 |
| Propane | — | — |
| Nitrogen | 1.9 | 0.6 |

Propane conversion could not be calculated as its peak area on the GC increased during reaction, indicating that more propane is being formed by the reaction than consumed.

As shown in Table 12, the initial gas phase carbon product selectivity to ethylene and acetylene was 21.8% and 24.1%, respectively, corresponding to production rates of 0.060 and 0.067 μmol/mL/s.

TABLE 12
Product selectivity (%) with standard deviation (%)
for the reaction with a feed gas composition of 28 mol
% CH₄, 4 mol % C₂H₆, 1 mol % C₃H₈, and 67 mol % N₂.

| | Selectivity (%) | Std. Dev. (%) |
|---|---|---|
| Ethylene | 21.8 | 1.6 |
| Acetylene | 24.1 | 0.6 |
| Propylene | 7.7 | 0.09 |
| n-Butane | 12.8 | 0.3 |
| 1-Butene | 1.2 | 0.01 |
| Isobutene | 1.9 | 0.1 |
| 1,3-Butadiene | 0.6 | 0.07 |
| cis/trans-2-Butene | 1.0 | 0.4 |
| C5 | 6.9 | 0.4 |
| C6 | 1.8 | 0.5 |
| C7 | 1.0 | 0.8 |
| Unknown | 19.2 | 1.4 |

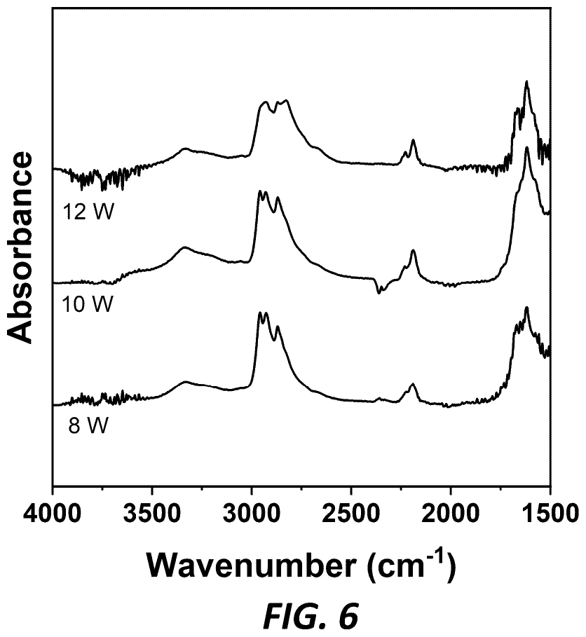

The reaction was initially 58.3% selective towards alkenes and alkynes in the gas phase. The carbon balance was calculated to be 94%. At this condition, the liquid production rate was 0.44 μg/mL/s for a 5-hour reaction. The in-line mass spectrometer identified additional products such as ammonia, hydrogen cyanide, acetonitrile, cyanamide, vinylamine, and 2-propanamaine. Attenuated total reflectance infrared spectroscopy (ATR-IR) of the liquid product showed features in the amine and nitrile regions (FIG. 6)

### Example 7

The experimental conditions of Example 1 were maintained (200° C., 50 mL/min), as well as the feed gas composition from Example 6 (28 mol % CH₄, 4 mol % C₂H₆, 1 mol % and 67 mol % N₂), while the power input was changed to 8 W. Here the initial conversions were 10.7%, 19.6%, and 1.5% for methane, ethane, and nitrogen, respectively (Table 13).

TABLE 13
Reactant conversion (%) with standard deviation
(%) for the reaction with a feed gas composition
of 28 mol % CH₄, 4 mol % C₂H₆, 1 mol % C₃H₈, and
67 mol % N₂ with a plasma input power of 8 W.

| | Conversion (%) | Std. Dev. (%) |
|---|---|---|
| Methane | 10.7 | 0.8 |
| Ethane | 19.6 | 0.2 |
| Propane | — | — |
| Nitrogen | 1.5 | 0.4 |

Propane conversion could not be calculated as its peak area on the GC increased during reaction, indicating that more propane is being formed by the reaction than consumed.

As shown in Table 14, the initial gas phase carbon product selectivity to ethylene and acetylene was 27.1% and 26.0%, respectively, corresponding to production rates of 0.056 and 0.053 μmol/mL/s.

TABLE 14
Product selectivity (%) with standard deviation
(%) for the reaction with a feed gas composition
of 28 mol % CH₄, 4 mol % C₂H₆, 1 mol % C₃H₈, and
67 mol % N₂ with a plasma input power of 8 W.

| | Selectivity (%) | Std. Dev. (%) |
|---|---|---|
| Ethylene | 27.1 | 0.6 |
| Acetylene | 26 | 0.5 |
| Propylene | 6.1 | 0.03 |
| n-Butane | 16.2 | 0.3 |
| 1-Butene | 0.6 | 0.1 |
| Isobutene | 1.1 | 0.06 |
| 1,3-Butadiene | 0.3 | 0.03 |
| cis/trans-2-Butene | 0.5 | 0.1 |
| C5 | 3.5 | 0.2 |
| C6 | 0.9 | 0.07 |
| C7 | 0.2 | 0.02 |
| Unknown | 17.5 | 1.0 |

The reaction was initially 61.7% selective towards alkenes and alkynes in the gas phase. The carbon balance was calculated as 95%. At this condition, the liquid production rate was 0.13 μg/mL/s for a 5-hour reaction. The in-line mass spectrometer identified additional products such as ammonia, hydrogen cyanide, acetonitrile, cyanamide, vinylamine, and 2-propanamaine. Attenuated total reflectance infrared spectroscopy (ATR-IR) of the liquid product showed features in the amine and nitrile regions (FIG. 6).

### Example 8

The experimental conditions of Example 1 were maintained (200° C., 50 mL/min), as well as the feed gas composition from Example 6 (28 mol % CH₄, 4 mol % C₂H₆, 1 mol % C₃H₈, and 67 mol % N₂), while the power input was changed to 12 W. Here the initial conversions were 14.1 mol %, 24.8 mol %, and 2.6 mol % for methane, ethane, and nitrogen, respectively (Table 15).

TABLE 15
Reactant conversion (%) with standard deviation
(%) for the reaction with a feed gas composition
of 28 mol % CH₄, 4 mol % C₂H₆, 1 mol % C₃H₈, and
67 mol % N₂ with a plasma input power of 12 W.

| | Conversion (%) | Std. Dev. (%) |
|---|---|---|
| Methane | 14.1 | 0.5 |
| Ethane | 24.8 | 3.0 |
| Propane | — | — |
| Nitrogen | 2.6 | 0.2 |

Propane conversion could not be calculated as its peak area on the GC increased during reaction, indicating that more propane is being formed by the reaction than consumed. As shown in Table 16, the initial gas phase carbon product selectivity to ethylene and acetylene were 35.3% and 23.5%, respectively, corresponding to production rates of 0.10 and 0.068 μmol/mL/s.

TABLE 16
Product selectivity (%) with standard deviation
(%) for the reaction with a feed gas composition
of 28 mol % CH₄, 4 mol % C₂H₆, 1 mol % C₃H₈, and
67 mol % N₂ with a plasma input power of 12 W.

| | Selectivity (%) | Std. Dev. (%) |
|---|---|---|
| Ethylene | 35.3 | 0.5 |
| Acetylene | 23.5 | 2.0 |
| Propylene | 8.6 | 1.3 |
| n-Butane | 12.2 | 0.2 |
| 1-Butene | 0.6 | 0.04 |
| Isobutene | 1.6 | 0.1 |
| 1,3-Butadiene | 0.2 | 0.07 |
| cis/trans-2-Butene | 0.4 | 0.06 |
| C5 | 2.5 | 0.05 |
| C6 | 0.4 | 0.1 |
| C7 | 0.2 | 0.07 |
| Unknown | 14.5 | 0.02 |

The reaction was initially 70.2% selective towards alkenes and alkynes in the gas phase. The carbon balance was calculated to be 96%. At this condition, the liquid production rate was 0.27 μg/mL/s for a 5-hour reaction. The in-line mass spectrometer identified additional products such as ammonia, hydrogen cyanide, acetonitrile, cyanamide, vinylamine, and 2-propanamaine. Attenuated total reflectance infrared spectroscopy (ATR-IR) of the liquid product showed features in the amine and nitrile regions (FIG. 6).

For reasons of completeness, various aspects of the technology are set out in the following numbered embodiments:

Embodiment 1. A shale gas processing system, comprising:
a reactor comprising:
   a first inlet in fluid communication with a shale gas source;
   a plasma zone in fluid communication with the first inlet, the plasma zone comprising:
      an inner electrode;
      an outer electrode; and
      an inner volume defined between the inner electrode and the outer electrode;
   an outlet in fluid communication with the plasma zone;
a collection vessel configured to receive fluid from the reactor outlet; and
a voltage supply and monitor system in electrical communication with the inner electrode and with the outer electrode.

Embodiment 2. The shale gas processing system according to embodiment 1, the reactor further comprising a second inlet in fluid communication with a nitrogen (N₂) gas source,
the first inlet and the second inlet being positioned at an upper portion of the reactor, and
the outlet being positioned at a lower portion of the reactor.

Embodiment 3. The shale gas processing system according to embodiment 1 or embodiment 2, the system further comprising a depressurization unit arranged to provide shale gas to the first inlet.

Embodiment 4. The shale gas processing system according to any one of embodiments 1-3, the plasma zone having a discharge gap of 0.1 mm to 150 mm; and
the plasma zone being cylindrical; and
the outer electrode annularly defining an exterior of the plasma zone.

Embodiment 5. The shale gas processing system according to any one of embodiments 1-4, the inner electrode comprising tungsten and the outer electrode comprising stainless steel.

Embodiment 6. The shale gas processing system according to any one of embodiments 1-5, the reactor further comprising a reactor temperature regulation unit arranged to maintain a predetermined temperature within the reactor.

Embodiment 7. The shale gas processing system according to any one of embodiments 1-6, wherein the reactor does not include catalyst material.

Embodiment 8. The shale gas processing system according to any one of embodiments 1-7, the system further comprising a gas chromatograph (GC) in fluid communication with the reactor outlet, the gas chromatograph (GC) comprising a thermal conductivity detector (TCD), a flame ionization detector (FID), and a photoionization detector (PID).

Embodiment 9. The shale gas processing system according to any one of embodiments 1-8, the voltage supply and monitor system comprising:
an alternating current (AC) power source in electrical communication with the inner electrode;
an oscilloscope;
a voltage attenuator in electrical communication with the AC power source and the oscilloscope; and
a monitor capacitor in electrical communication with the outer electrode and the oscilloscope.

Embodiment 10. A method for processing shale gas, the method comprising:
providing shale gas at a first flowrate to a first inlet of a reactor;
providing a voltage to an inner electrode disposed within a plasma zone of the reactor;
thereby generating a plasma in the plasma zone across a discharge gap; and
collecting products from an outlet of the reactor.

Embodiment 11. The method according to embodiment 10, further comprising providing nitrogen (N₂) gas at a second flowrate to a second inlet of the reactor, wherein the first flowrate is 5 standard cubic centimeters per minute (cm³/min) to 11,304 cm³/min and the second flowrate is 1 cm³/min to 10,174 cm³/min.

Embodiment 12. The method according to embodiment 10 or 11, further comprising, before providing the shale gas to the first inlet of the reactor, decompressing the shale gas to a pressure no greater than 0.3 Megapascal (MPa).

Embodiment 13. The method according to any one of embodiments 10-12, wherein the plasma zone is maintained at a temperature of 25° C. to 250° C., and the discharge gap is 0.1 mm to 150 mm.

Embodiment 14. The method according to any one of embodiments 10-13, wherein the voltage provided is no less than 6 kV and no greater than 9 kV, and wherein the voltage is provided at a frequency of 2 kHz to 700 kHz.

Embodiment 15. The method according to any one of embodiments 10-14, wherein the voltage is provided from an AC power source.

Embodiment 16. The method according to any one of embodiments 10-15, further comprising, before providing the voltage, attenuating an output signal from the AC power source, and, while providing the voltage, monitoring the voltage across a capacitor.

Embodiment 17. The method according to any one of embodiments 10-16, further comprising, after collecting the products, recycling unreacted gases back to the first inlet of the reactor.

Embodiment 18. A method of preparing nitrogen (N)-containing compounds, the method comprising:
providing a gas composition to a reactor;
providing a voltage to an inner electrode disposed within a plasma zone of the reactor;
thereby generating a plasma in the plasma zone across a discharge gap;
collecting products from an outlet of the reactor, the products comprising nitrogen (N)-containing compounds.

Embodiment 19. The method according to embodiment 18, the gas composition comprising, by mol %:
7% to 94% methane (CH₄),
2% to 20% ethane (C₂H₆),
1% to 11% propane (C₃H₈), and
2% to 90% N₂.

Embodiment 20. The method according to embodiment 18 or 19, the nitrogen (N)-containing compounds comprising:
a combination of nitrogen (N), carbon (C), and hydrogen (H) atoms; or
a combination of nitrogen (N) and hydrogen (H) atoms.

1. A shale gas processing system, comprising:
a reactor comprising:
a first inlet in fluid communication with a shale gas source;
a plasma zone in fluid communication with the first inlet, the plasma zone comprising:
an inner electrode;
an outer electrode; and
an inner volume defined between the inner electrode and the outer electrode;
an outlet in fluid communication with the plasma zone;
a collection vessel configured to receive fluid from the reactor outlet; and
a voltage supply and monitor system in electrical communication with the inner electrode and with the outer electrode.

2. The shale gas processing system according to claim 1, the reactor further comprising a second inlet in fluid communication with a nitrogen (N₂) gas source,
the first inlet and the second inlet being positioned at an upper portion of the reactor, and
the outlet being positioned at a lower portion of the reactor.

3. The shale gas processing system according to claim 1, the system further comprising a depressurization unit arranged to provide shale gas to the first inlet.

4. The shale gas processing system according to claim 1, the plasma zone having a discharge gap of 0.1 mm to 150 mm; and
the plasma zone being cylindrical; and
the outer electrode annularly defining an exterior of the plasma zone.

5. The shale gas processing system according to claim 1, the inner electrode comprising tungsten and the outer electrode comprising stainless steel.

6. The shale gas processing system according to claim 1, the reactor further comprising a reactor temperature regulation unit arranged to maintain a predetermined temperature within the reactor.

7. The shale gas processing system according to claim 1, wherein the reactor does not include catalyst material.

8. The shale gas processing system according to claim 1, the system further comprising a gas chromatograph (GC) in fluid communication with the reactor outlet, the gas chromatograph (GC) comprising a thermal conductivity detector (TCD), a flame ionization detector (FID), and a photoionization detector (PID).

9. The shale gas processing system according to claim 1, the voltage supply and monitor system comprising:
an alternating current (AC) power source in electrical communication with the inner electrode;
an oscilloscope;
a voltage attenuator in electrical communication with the AC power source and the oscilloscope; and
a monitor capacitor in electrical communication with the outer electrode and the oscilloscope.

10. A method for processing shale gas, the method comprising:
providing shale gas at a first flowrate to a first inlet of a reactor;
providing a voltage to an inner electrode disposed within a plasma zone of the reactor;
thereby generating a plasma in the plasma zone across a discharge gap; and
collecting products from an outlet of the reactor.

11. The method according to claim 10, further comprising providing nitrogen (N₂) gas at a second flowrate to a second inlet of the reactor, wherein the first flowrate is 5 standard cubic centimeters per minute (cm³/min) to 11,304 cm³/min and the second flowrate is 1 cm³/min to 10,174 cm³/min.

12. The method according to claim 10, further comprising, before providing the shale gas to the first inlet of the reactor, decompressing the shale gas to a pressure no greater than 0.3 Megapascal (MPa).

13. The method according to claim 10, wherein the plasma zone is maintained at a temperature of 25° C. to 250° C., and the discharge gap is 0.1 mm to 150 mm.

14. The method according to claim 10, wherein the voltage provided is no less than 6 kV and no greater than 9 kV, and wherein the voltage is provided at a frequency of 2 kHz to 700 kHz.

15. The method according to claim 10, wherein the voltage is provided from an AC power source.

16. The method according to claim 15, further comprising, before providing the voltage, attenuating an output signal from the AC power source, and, while providing the voltage, monitoring the voltage across a capacitor.

17. The method according to claim 10, further comprising, after collecting the products, recycling unreacted gases back to the first inlet of the reactor.

18. A method of preparing nitrogen (N)-containing compounds, the method comprising:
providing a gas composition to a reactor;
providing a voltage to an inner electrode disposed within a plasma zone of the reactor;
thereby generating a plasma in the plasma zone across a discharge gap;
collecting products from an outlet of the reactor, the products comprising nitrogen (N)-containing compounds.

19. The method according to claim 18, the gas composition comprising, by mol %:
7% to 94% methane (CH₄),
2% to 20% ethane (C₂H₆),
1% to 11% propane (C₃H₈), and
2% to 90% N₂.

20. The method according to claim 18, the nitrogen (N)-containing compounds comprising:
a combination of nitrogen (N), carbon (C), and hydrogen (H) atoms; or
a combination of nitrogen (N) and hydrogen (H) atoms.
